(19) United States
(12) Patent Application Publication
(10) Pub. No.: US 2013/0084213 A1
(43) Pub. Date: Apr. 4, 2013

(54) SAMPLE PROCESSING APPARATUS

(71) Applicant: Sysmex Corporation, Kobe-shi (JP)

(72) Inventors: Ken NISHIKAWA, Kobe-shi (JP); Konobu KIMURA, Kobe-shi (JP); Yuichi HAMADA, Kobe-shi (JP)

(73) Assignee: SYSMEX CORPORATION, Kobe-shi (JP)

(21) Appl. No.: 13/630,436

(22) Filed: Sep. 28, 2012

(30) Foreign Application Priority Data
Sep. 30, 2011 (JP) 2011-216323

(51) Int. Cl. G01N 33/50

(52) U.S. Cl. 422/73

(57) ABSTRACT

A sample processing apparatus comprises a sample processing unit for processing a sample, a control unit for accepting a stop instruction to stop the operation of the sample processing unit, and an output unit. The control unit is configured to control the output unit to output consumable good information related to lack of the consumable good that occurs if the consumable good runs out before completion of next starting of the sample processing unit when accepting the stop instruction.

### RELATED APPLICATIONS

This application claims priority under 35 U.S.C. §119 to Japanese Patent Application No. 2011-216323 filed on Sep. 30, 2011, the entire content of which is hereby incorporated by reference.

1. Field of the Invention

The present invention relates to a sample processing apparatus for processing a sample collected from humans or animals such as blood sample, urine sample, or the like.

2. Background

A sample processing apparatus for processing blood or urine such as blood cell counting apparatus, blood coagulation measurement apparatus, immune analyzer, biochemical analyzer, urine analyzer, and the like is known. In the sample processing apparatus, consumable goods such as reagent, cuvette, pipette tip, and the like are normally used.

In the sample processing apparatus, washing is generally carried out in a startup operation (see e.g., Japanese Unexamined Patent Publication No. 2010-107398). In the washing operation, consumable goods such as washing fluid and reagent are used.

However, in the analyzer disclosed in patent document 1, a case where the consumable goods run out in the startup operation is not taken into consideration. Therefore, if the consumable goods run out during the execution of the startup operation, the startup operation is interrupted, which startup operation cannot be resumed until replacement or restock of the consumable good is completed thus delaying the start of the sample process as a result.

### SUMMARY OF THE INVENTION

The scope of the present invention is defined solely by the appended claims, and is not affected to any degree by the statements within this summary.

An aspect of the present invention is a sample processing apparatus comprising a sample processing unit for processing a sample, a control unit for accepting a stop instruction to stop the operation of the sample processing unit, and an output unit. The control unit is configured to control the output unit to output consumable good information related to lack of the consumable good that occurs if the consumable good runs out before completion of next starting of the sample processing unit when accepting the stop instruction.

### BRIEF DESCRIPTION OF THE DRAWINGS

FIG. 1 is a perspective view showing an overall configuration of a sample processing apparatus according to a first embodiment;

FIG. 2 is a block diagram showing a configuration of a measurement unit arranged in the sample processing apparatus according to the first embodiment;

FIG. 3A is a fluid circuit diagram showing a configuration of a measurement mechanism arranged in the measurement unit;

FIG. 3B is a fluid circuit diagram showing a configuration of the measurement mechanism arranged in the measurement unit;

FIG. 4 is a block diagram showing a configuration of an information processing unit arranged in the sample processing apparatus according to the first embodiment;

FIG. 5 is a schematic view showing a configuration of reagent remaining amount information;

FIG. 6 is a flowchart showing an operation procedure of the sample processing apparatus in RBC/PLT measurement and HGB measurement;

FIG. 7 is a flowchart showing an operation procedure of the sample processing apparatus in CBC+DIFF measurement;

FIG. 8 is a view showing a startup setting screen;

FIG. 9 is a flowchart showing a flow of shutdown operation of the sample processing apparatus according to the first embodiment;

FIG. 10 is a view showing a first notification screen;

FIG. 11 is a flowchart showing a procedure of a reagent replacing process in S307 and S313 of FIGS. 9, and S713 of FIG. 15;

FIG. 12 is a flowchart showing a procedure of a reagent usage amount determining process in S309 of FIG. 9 and S709 of FIG. 15;

FIG. 13 is a view showing a second notification screen;

FIG. 14 is a flowchart showing a flow of a startup operation of the sample processing apparatus according to the first embodiment; and

FIG. 15 is a flowchart showing a shutdown operation of the sample processing apparatus according to a second embodiment.

### DETAILED DESCRIPTION OF THE PREFERRED EMBODIMENTS

The preferred embodiments of the present invention will be described hereinafter with reference to the drawings.

Preferred embodiments of the present invention will be hereinafter described with reference to the drawings.

### First Embodiment

### Configuration of Sample Processing Apparatus

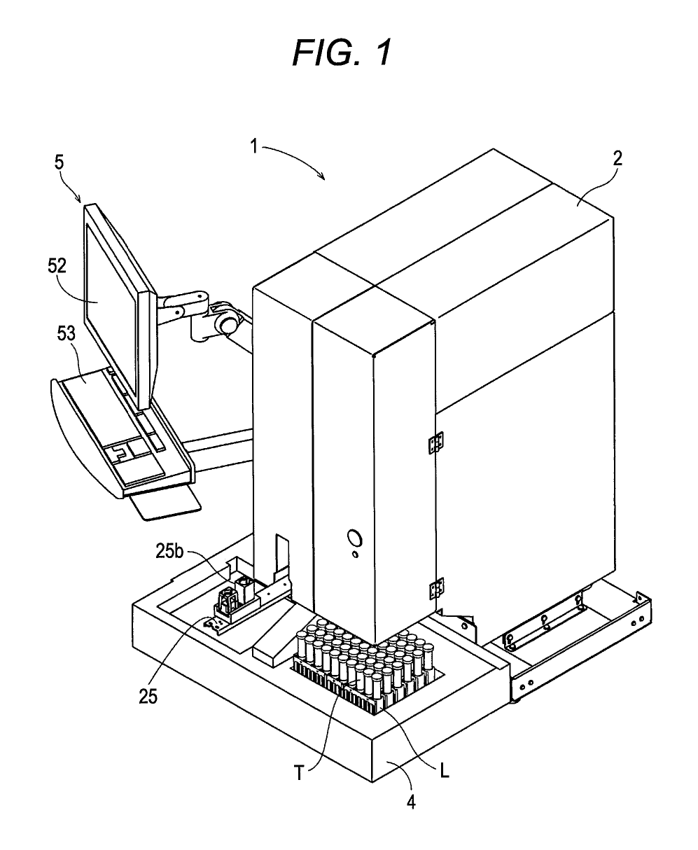

FIG. 1 is a perspective view showing an overall configuration of a sample processing apparatus according to the present embodiment. A sample processing apparatus 1 according to the present embodiment is a multi-item blood cell analyzer for detecting white blood cells, red blood cells, blood platelets, and the like contained in a blood sample, and counting each blood cell. As shown in FIG. 1, the blood analyzer 1 includes a measurement unit 2, a sample transport unit 4 arranged on a front side of the measurement unit 2, and an information processing unit 5 capable of controlling the measurement unit 2 and the sample transport unit 4.

The sample processing apparatus 1 transports a sample rack holding a plurality of sample tubes with the sample transport unit 4, aspirates the sample from the sample tube with the measurement unit 2, and analyzes the relevant sample. The sample tube T has a tubular shape with the upper end opened. The blood sample collected from a patient is contained inside, and the opening at the upper end is sealed with a lid. The sample tube T is made from glass or synthetic resin having translucency, so that that blood sample inside is visible. A barcode label is attached to a side surface of the sample tube T. A barcode indicating a sample ID is printed on the barcode label. The sample rack L can hold ten sample tubes T in a line. Each sample tube T is held in a perpendicular state (upright state) in the sample rack L. A barcode label is attached to a side surface of the sample rack L. A barcode indicating a rack ID is printed on the barcode label.

### Configuration of Measurement Unit

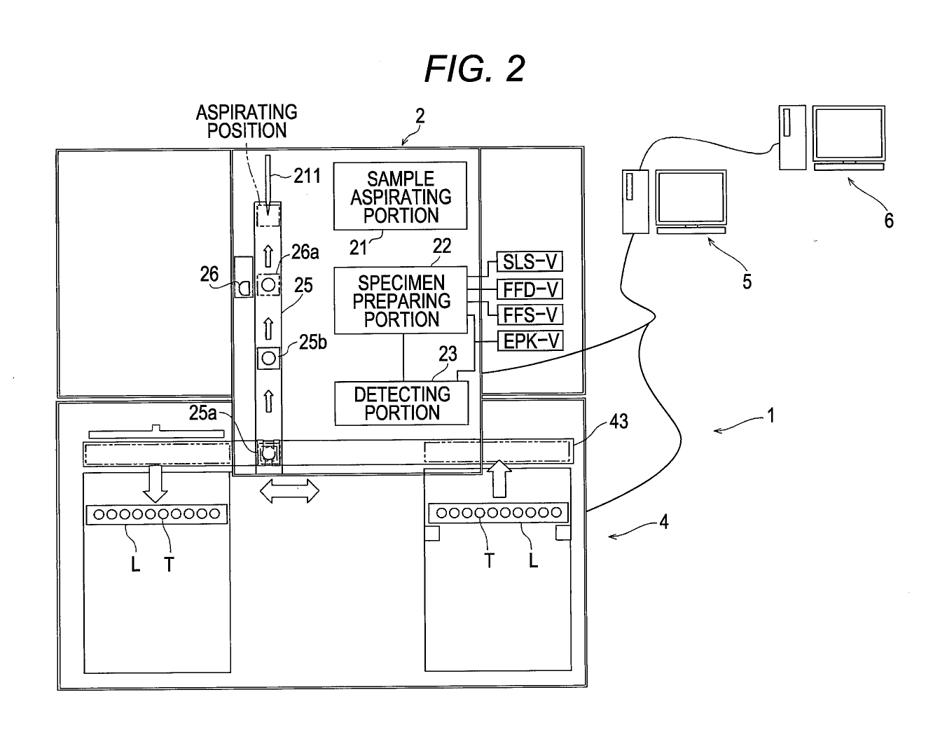

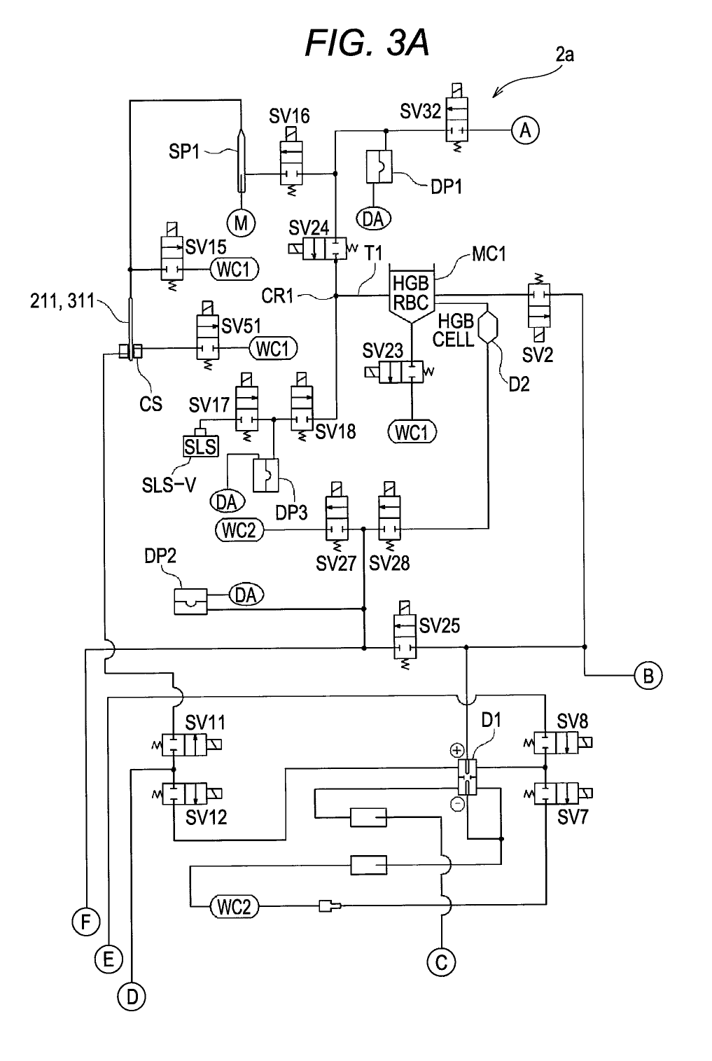

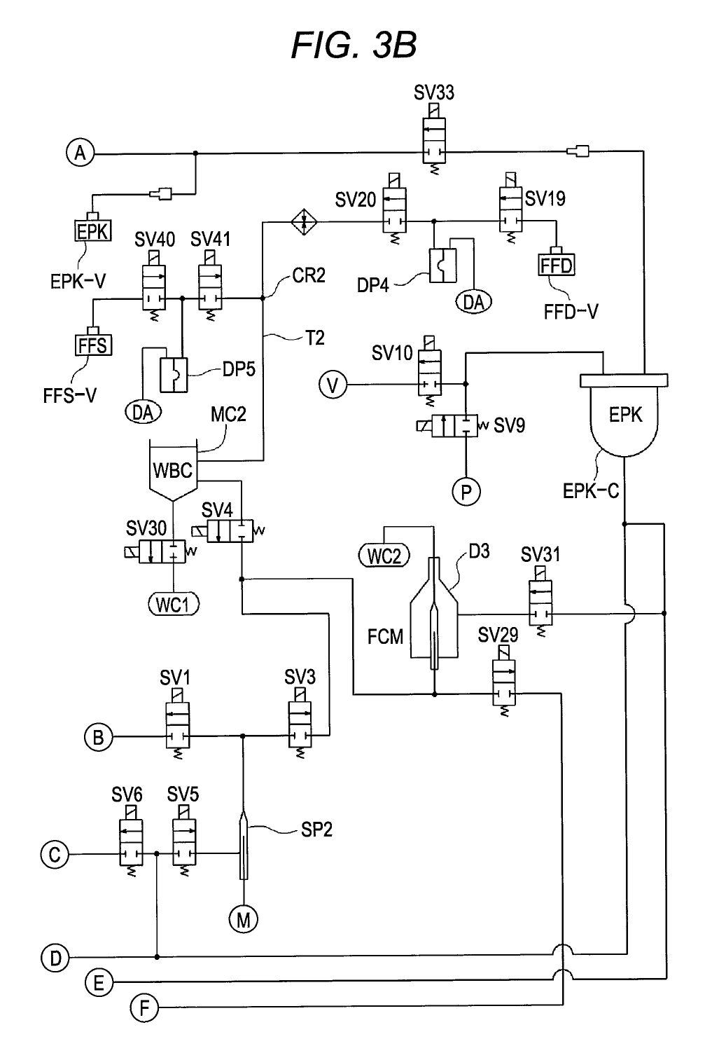

A configuration of the measurement unit will now be described. FIG. 2 is a block diagram showing a configuration of the measurement unit, and FIG. 3A and FIG. 3B are fluid circuit diagrams showing a configuration of a measurement mechanism arranged in the measurement unit. As shown in FIG. 2, the measurement unit 2 includes a measurement mechanism 2a with a sample aspirating portion 21 for aspirating blood, which is a sample, from a sample tube (blood collecting tube) T, a specimen preparing portion 22 for preparing a measurement specimen used in the measurement from the blood aspirated by the sample aspirating portion 21, and a detecting portion 23 for detecting the blood cells from the measurement specimen prepared by the specimen preparing portion 22. The measurement unit 2 further includes a take-in port for taking in the sample tube T accommodated in the sample rack L transported by a rack transporting portion 43 of the sample transport unit 4 inside the measurement unit 2, and a sample tube transporting portion 25 for taking in the sample tube T from the sample rack L into the measurement unit 2, and transporting the sample tube T to an aspirating position by the sample aspirating portion 21.

First, the configuration of the sample tube transporting portion 25 will be described. The sample tube transporting portion 25 includes a hand portion 25a that can grip the sample tube T. The sample tube T accommodated in the sample rack L is gripped by the hand portion 25a, the hand portion 25a is moved upward in this state to take out the sample tube T from the sample rack L, and the hand portion 25a is oscillated. The sample in the sample tube thus can be stirred.

The sample tube transporting portion 25 includes a sample tube setting portion 25b having a hole to which the sample tube T can be inserted. The sample tube T gripped by the hand portion 25a is set in the sample tube setting portion 25b. The sample tube setting portion 25b can be moved horizontally in the Y direction by the power of the stepping motor (not shown).

A barcode reading portion 26 is arranged inside measurement unit 2. The sample tube setting portion 25b can be moved to a barcode reading position 26a near the barcode reading portion 26 and the aspirating position 21a by the sample aspirating portion 21. When the sample tube setting portion 25b is moved to the barcode reading position 26a, the sample barcode is read by the barcode reading portion 26. When the sample tube setting portion 25b is moved to the aspirating position, the sample is aspirated from the set sample tube T by the sample aspirating portion 21.

As shown in FIG. 2, an aspirating tube 211 shown in FIG. 3A is arranged at a distal end of the sample aspirating portion 21. The sample aspirating portion 21 includes a whole blood aspirating syringe pump SP1. The sample aspirating portion 21 can move in a vertical direction, and is configured such that when moved to the lower side, the aspirating tube passes through the lid of the sample tube T transported to the aspirating position to aspirate the blood inside.

The specimen preparing portion 22 includes a first mixing chamber MC1 and a second mixing chamber MC2 (see FIG. 3A and FIG. 3B). The aspirating tube 211 aspirates the whole blood sample of a predetermined amount from the sample tube T with a whole blood aspirating syringe pump SP1, the aspirated sample is then transferred to the positions of the first mixing chamber MC1 and the second mixing chamber MC2, and a whole blood sample of a predetermined amount is distributed and supplied to the respective chambers MC1, MC2 by the whole blood aspirating syringe pump SP1.

A reagent tube containing the reagent can be installed in the measurement unit 2, and the reagent tube can be connected to a fluid circuit. Specifically, the reagent tube used in the present embodiment is a diluted solution tube EPK-V for containing the diluted solution (washing fluid) EPK, a hemoglobin hemolytic agent tube SLS-V for containing a hemoglobin hemolytic agent SLS, a white blood cell classifying hemolytic agent tube (common reagent tube) FFD-V for containing the white blood cell classifying hemolytic agent FFD for dissolving red blood cells, and a white blood cell classifying stain fluid tube (dedicated reagent tube) FFS-V for containing the white blood cell classifying stain fluid FFS (see FIG. 2, FIG. 3A, and FIG. 3B).

The measurement unit 2 includes a diluted solution chamber EPK-C for temporarily accommodating the diluted solution (washing fluid) EPK. The diluted solution chamber EPK-C is connected to a diluted solution tube EPK-V, so that the diluted solution can be supplied from the diluted solution tube EPK-V. In the present embodiment, the capacity of the diluted solution chamber EPK-C is less than one measurement. In other words, when performing the measurement, the diluted solution stored in the diluted solution chamber EPK-C may not be enough, and the measurement may need to be carried out while supplying the diluted solution from the diluted solution tube EPK-V to the diluted solution chamber EPK-C.

The diluted solution chamber EPK-C and the hemolytic agent tube SLS-V are connected to be able to supply the reagent to the first mixing chamber MC1. In other words, the diluted solution can be supplied from the diluted solution chamber EPK-C to the first mixing chamber MC1 by a diluted solution supply (EPK) diaphragm pump DP1, which EPK diaphragm pump DP1 configures a reagent supplying portion for the diluted solution. The diaphragm pumps DP1 to DP5 shown in FIG. 3A and FIG. 3B are connected to a positive pressure source and a negative pressure source through an electromagnetic valve, and are driven by the positive pressure source and the negative pressure source.

The hemolytic agent can be supplied from the hemolytic agent tube SLS-V to the first mixing chamber MC1 by a hemolytic agent supply (SLS) diaphragm pump DP3, which SLS diaphragm pump DP3 configures the reagent supplying portion for the hemolytic agent.

The hemolytic agent tube FFD-V and the stain fluid tube FFS-V are connected to be able to supply the reagent to the second mixing chamber MC2. In other words, the hemolytic agent can be supplied from the hemolytic agent tube FFD-V to the second mixing chamber MC2 by a hemolytic agent (FFD) diaphragm pump DP4, which FFD diaphragm pump DP4 configures the reagent supplying portion for the hemolytic agent.

The stain fluid can be supplied from the stain fluid tube FFS-V to the second mixing chamber MC2 by a stain fluid (FFS) diaphragm pump DP5, which FFS diaphragm pump DP5 configures the reagent supplying portion for the stain fluid.

A reagent supply path from the diluted solution chamber EPK-C to the first mixing chamber MC1 and a reagent supply path from the hemolytic agent tube SLS-V to the first mixing chamber MC1 are merged at a merging point CR1 in the middle, and a reagent supply path T1 common to both reagents is connected to the first mixing chamber MC1 (see FIG. 3A). A reagent supply path from the hemolytic agent tube FFD-V to the second mixing chamber MC2 and a reagent supply path from the stain fluid tube FFS-V to the second mixing chamber MC2 are also merged at a merging point CR2 in the middle, and a reagent supply path T2 common to both reagents is connected to the second mixing chamber MC2 (see FIG. 3B). The reagent supply paths T1, T2 may be arranged for every reagent. In other words, two reagent supply ports may be arranged for each chamber MC1, MC2.

The detecting portion 23 includes a first detector D1 for carrying out a measurement related to red blood cells and blood platelets, a second detector D2 for carrying out a measurement related to hemoglobin, and a third detector D3 for carrying out a measurement related to white blood cells.

The first mixing chamber MC1 is a portion for preparing a measurement specimen to carry out analysis related to the red blood cells, blood platelets, and hemoglobin, and the measurement specimen prepared in the first mixing chamber MC1 is used for the measurement in the first detector D1 and the second detector D2. The second mixing chamber MC2 is a portion for preparing a specimen to carry out analysis related to the white blood cells, and the specimen prepared in the second mixing chamber MC2 is used for the measurement in the third detector D3.

The first detector D1 is configured as the RBC/PLT detector for carrying out RBC measurement (measurement of number of red blood cells) and PLT measurement (measurement of number of blood platelets). The RBC/PLT detector D1 can carry out the measurement of the RBC and the PLT through a sheath flow DC detection method.

The second detector D2 is configured as an HGB detector for carrying out HGB measurement (measurement of amount of hemoglobin in the blood). The HGB detector D2 can carry out the HGB measurement by the SLS-hemoglobin method.

The third detector D3 is configured as an optical detector for carrying out WBC measurement (counting number of white blood cells) and DIFF measurement (white blood cell classification). The optical detector D3 is configured to carry out the detection of the WBC (white blood cells), NEUT (neutrophil cells), LYMPH (lymphocytes), EO (eosinocytes), BASO (basocytes), and MONO (monocytes) through the flow cytometry method using the semiconductor laser. The measurement of the measurement specimen in which the stain fluid, the hemolytic agent, and the diluted solution are mixed is carried out by the third detector D3, and the measurement data obtained as a result is subjected to the analysis process by the information processing unit 5 to carry out the measurement of the NEUT, LYMPH, EO, BASO, MONO, and WBC.

The third detector D3 includes a flow cell, and is adapted to irradiate a semiconductor laser light on the measurement specimen sent into the flow cell, and receive the forward scattered light, the lateral scattered light, and the lateral fluorescent light generated at that time to detect the forward scattered light intensity, the lateral scattered light intensity, and the lateral florescent light intensity. The measurement data containing each optical information of the forward scattered light intensity, the lateral scattered light intensity, and the lateral fluorescent light intensity obtained in such manner is transmitted from the measurement unit 2 to the information processing unit 5, and analyzed by the information processing unit 5.

### Configuration of Sample Transport Unit

A configuration of the sample transport unit 4 will now be described. As shown in FIG. 1, the sample transport unit 4 is arranged on the front side of the measurement unit 2 of the sample processing device 1. Such sample transport unit 4 can transport the sample rack L to supply the sample to the measurement unit 2.

The sample transport unit 4 includes a pre-analysis rack holding portion 41 for temporarily holding a plurality of sample racks L that holds the sample tube T containing the sample before undergoing the analysis, a post-analysis rack holding portion 42 for temporarily holding the plurality of sample racks L that holds the sample tube T from which the sample is aspirated by the measurement unit 2, and a rack transporting portion 43 for linearly moving horizontally the sample rack L in a direction of an arrow X in the figure, and transporting the sample rack L received from the pre-analysis rack holding portion 41 to the post-analysis rack holding portion 42 to supply the sample to the measurement unit 2. The sample rack L set in the pre-analysis rack holding portion 41 is moved in the X direction by the rack transporting portion 43, and the sample in the sample tube held in the sample rack L is aspirated at the aspirating position to carry out the sample measurement by the measurement unit 2. After the sample is aspirated from all the sample tubes held in the sample rack L, the sample rack L is transferred to the post-analysis rack holding portion 41.

### Configuration of Information Processing Unit

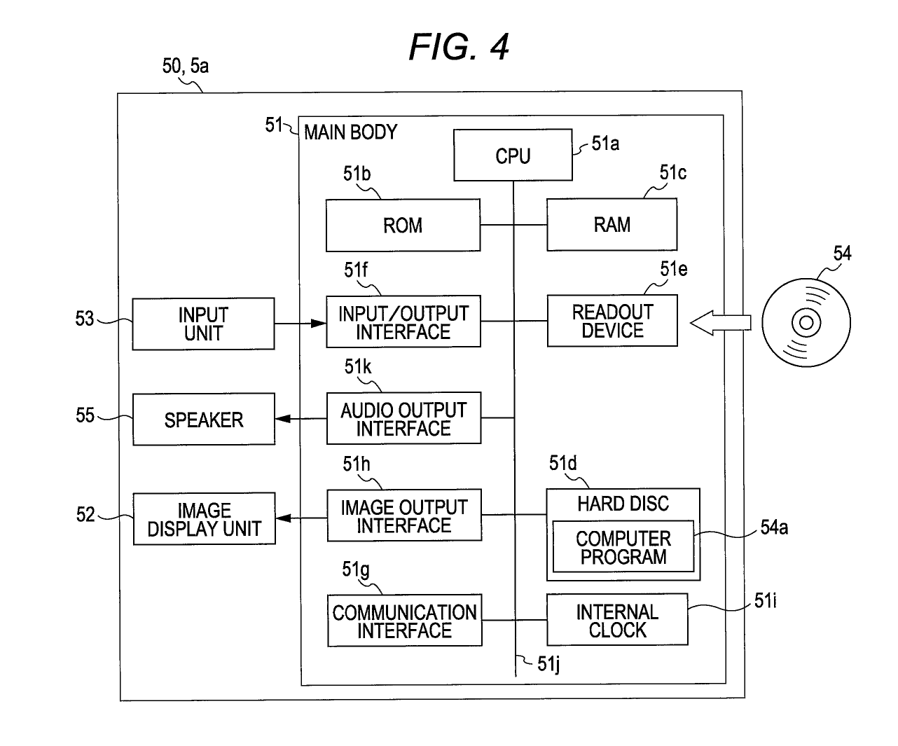

A configuration of the information processing unit 5 will now be described. The information processing unit 5 is configured by a computer. FIG. 4 is a block diagram showing a configuration of the information processing unit 5. As shown in FIG. 4, a computer 5a includes a main body 51, a display unit 52, an input unit 53, and a speaker 55. The main body 51 includes a CPU 51a, a ROM 51b, a RAM 51c, a hard disc 51d, a readout device 51e, an input/output interface 51f, a communication interface 51g, an image output interface 51h, an internal clock 51i, and an audio output interface 51k, where the CPU 51a, the ROM 51b, the RAM 51c, the hard disc 51d, the readout device 51e, the input/output interface 51f, the communication interface 51g, the image output interface 51h, the internal clock 51i, and the audio output interface 51k are connected by a bus 51j.

The CPU 51a is capable of executing the computer program loaded in the RAM 51c. The computer 5a functions as the information processing unit 5 when the CPU 51a executes the computer program 54a for sample analysis and control for the measurement unit 2 and the sample transport unit 4, as described later.

The ROM 51b is configured by mask ROM, PROM, EPROM, EEPROM, or the like, and is recorded with computer programs to be executed by the CPU 51a, data used for the same, and the like.

The RAM 51c is configured by SRAM, DRAM, and the like. The RAM 51c is used to read out the computer program 54a recorded on the hard disc 51d. The RAM 51c is used as a work region of the CPU 51a when the CPU 51a executes the computer programs.

The hard disc 51d is installed with various computer programs to be executed by the CPU 51a such as operating system and application program, as well as data used in executing the computer program. The computer program 54a, to be described later, is also installed in the hard disc 51d. The computer program 54a is an event driven computer program.

The read-out device 51e is configured by flexible disc drive, CD-ROM drive, DVD-ROM drive, and the like, and is able to read out computer programs and data recorded on a portable recording medium 54. The portable recording medium 54 stores the computer program 54a for causing the computer to function as the information processing unit 5, and the computer 5a reads out the computer program 54a from the portable recording medium 54 and installs the computer program 54a in the hard disc 51d.

The computer program 54a is not only provided by the portable recording medium 54, and may be provided through an electric communication line (wired or wireless) from external devices which are communicably connected to the computer 5a via the electric communication line. For example, the computer program 54a may be stored in a hard disk of the server computer on the Internet, where the computer 5a accesses the server computer and downloads the computer program to install the same in the hard disc 51d.

The hard disc 51d is installed with a multi-task operating system such as Windows (registered trademark) manufactured and sold by Microsoft, for example. In the following description, the computer program 54a according to the present embodiment is assumed to operate on the operating system.

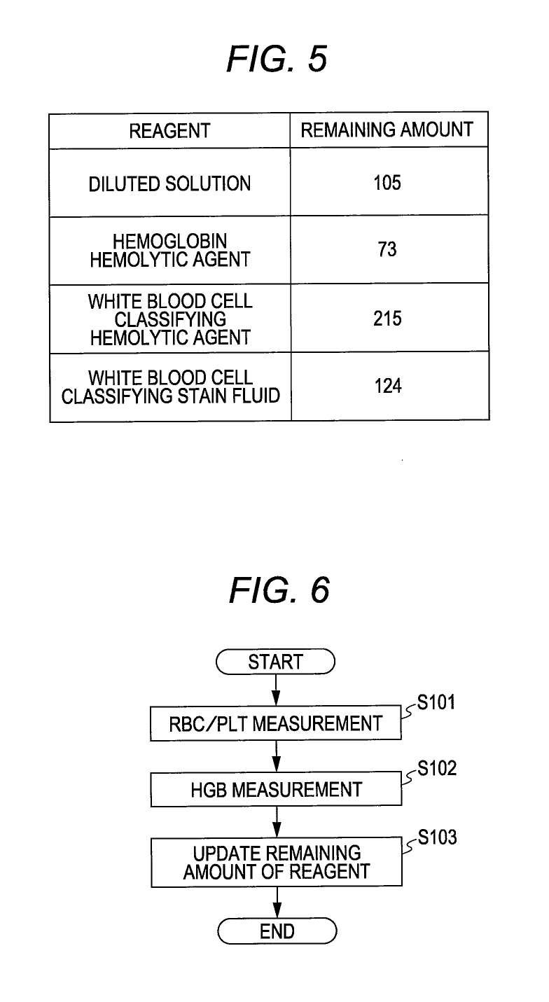

The hard disc 51d stores reagent remaining amount information 54b and setting information 54c. FIG. 5 is a schematic view showing a configuration of the reagent remaining amount information 54d. The remaining amount of the reagent is stored for the reagent remaining amount information 54d for every type of reagent (diluted solution, hemoglobin hemolytic agent, white blood cell classifying hemolytic agent, and white blood cell classifying stain fluid). The remaining amount of the reagent is represented by the number of measurements indicating how many more measurements can be carried out.

The input/output interface 51f includes a serial interface such as USB, IEEE1394, and RS-232C; a parallel interface such as SCSI, IDE, and IEEE1284; and an analog interface such as D/A converter and A/D converter. The input/output interface 51f is connected with the input unit 53 including a keyboard and a mouse, so that the operator can input data to the computer 5a by using the input unit 53. The input/output interface 51f is connected to the measurement unit 2 and the sample transport unit 4. The information processing unit 5 thus can control the measurement unit 2 and the sample transport unit 4.

The communication interface 51g is, for example, Ethernet (registered trademark) interface. The communication interface 51g is connected to a host computer (not shown) through the LAN. The computer 5a transmits and receives data with the host computer connected to the LAN using a predetermined communication protocol by means of the communication interface 51g.

The image output interface 51h is connected to the display unit 52 configured by LCD, CRT, or the like, and is configured to output an image signal corresponding to the image data provided from the CPU 51a to the display unit 52. The display 52 displays the image (screen) according to the input image signal.

The audio output interface 51k is connected to a speaker 55 to output an audio signal corresponding to the audio data provided from the CPU 51a to the speaker 55. The speaker 55 outputs audio according to the input audio signal.

The internal clock 51i can output current time. The CPU 51a can acquire the current time from the internal clock 51i.

### Measurement Operation of Sample Processing Apparatus 1

The operation of the sample processing apparatus 1 according to the present embodiment will now be described.

### Sample Measuring Operation

First, the sample measuring operation of the sample processing apparatus 1 according to the present embodiment will be described. The sample processing apparatus 1 can execute the RBC/PLT measurement using the first detector D1, the HGB measurement using the second detector D2, and the CBC+DIFF measurement using the third detector D3.

### RBC/PLT Measurement, HGB Measurement

First, the RBC/PLT measurement and the HGB measurement will be described. The RBC/PLT measurement and the HGB measurement are carried out in parallel to the CBC+DIFF measurement described above.

FIG. 6 is a flowchart showing an operation procedure of the sample processing apparatus 1 in the RBC/PLT measurement and the HGB measurement. First, the CPU 51a of the information processing unit 5 causes the measurement unit 2 to execute the RBC/PLT measurement (step S101).

In the RBC/PLT measurement, the diluted solution EPK is supplied to the first mixing chamber MC1 by the diluted solution (EPK) diaphragm pump DP1, and the whole blood sample of the sample tube T is aspirated by a constant amount with the aspirating tube 211 and discharged to the first mixing chamber MC1. The whole blood sample (4 μL) and the diluted solution EPK (2 mL) are thus stirred in the first mixing chamber MC1 to prepare the RBC/PLT measurement mixed specimen. One part of the RBC/PLT measurement mixed specimen is then supplied to the RBC/PLT detector D1 to carry out the RBC/PLT measurement.

An output signal (analog signal) output by such RBC/PLT detector D1 is converted to a digital signal by an A/D converter (not shown), subjected to a predetermined signal processing by a signal processing circuit (not shown) to convert digital data to measurement data, and the measurement data is transmitted to the information processing unit 5. The CPU 51a of the information processing unit 5 executes a predetermined analyzing process on the measurement data to generate analysis result data including the numerical value data of the RBC and the PLT, and stores the analysis result data in the hard disc 51d.

After the RBC/PLT measurement, the CPU 51a causes the measurement unit 2 to execute the HGB measurement (step S102). 1 mL of RBC/PLT measurement mixed specimen exists in the first mixing chamber MC1 as a remaining specimen even after the RBC/PLT measurement is completed. The hemolytic agent SLS is further supplied to the first mixing chamber MC1 containing the remaining specimen to adjust the HGB measurement mixed specimen. The hemolytic agent SLS and the RBC/PLT measurement mixed specimen are thus stirred, and the HGB measurement mixed specimen in which the hemolytic agent SLS (0.5 mL) is mixed to the RBC/PLT measurement mixed specimen (1.0 mL) is prepared. It is then left untouched for a predetermined time to wait for the reaction of the HGB measurement mixed specimen. The HGB measurement mixed specimen is then charged to the HGB detector D2, and the HGB measurement is carried out.

The output signal (analog signal) output by the HGB detector D2 is converted to a digital signal by an A/D converter (not shown), subjected to a predetermined signal processing by a signal processing circuit (not shown) to convert digital data to measurement data, and the measurement data is transmitted to the information processing unit 5. The CPU 51a of the information processing unit 5 executes a predetermined analyzing process on the measurement data to generate analysis result data including the numerical value data of the HGB, and stores the analysis result data in the hard disc 51d.

After executing the RBC/PLT measurement and the HGB measurement described above, the CPU 51a decrements the remaining amount of the reagent (diluted solution, hemoglobin hemolytic agent) used in the RBC/PLT measurement and the HGB measurement by one to update the reagent remaining amount information 54c (step S103), and terminates the process.

### CBC+DIFF Measurement

Next, the CBC+DIFF measurement will be described. In the CBC+DIFF measurement, the sample processing apparatus 1 mixes the whole blood sample (11 μL), the white blood cell classifying hemolytic agent (1 mL), and the white blood cell classifying stain fluid (20 μL) to prepare the CBC+DIFF measurement specimen, and measures the CBC+DIFF measurement specimen with the optical detector D3 through the flow cytometric method. In this measurement, the measurement on the number of white blood cells and the measurement of the five classifications of the white blood cells are carried out.

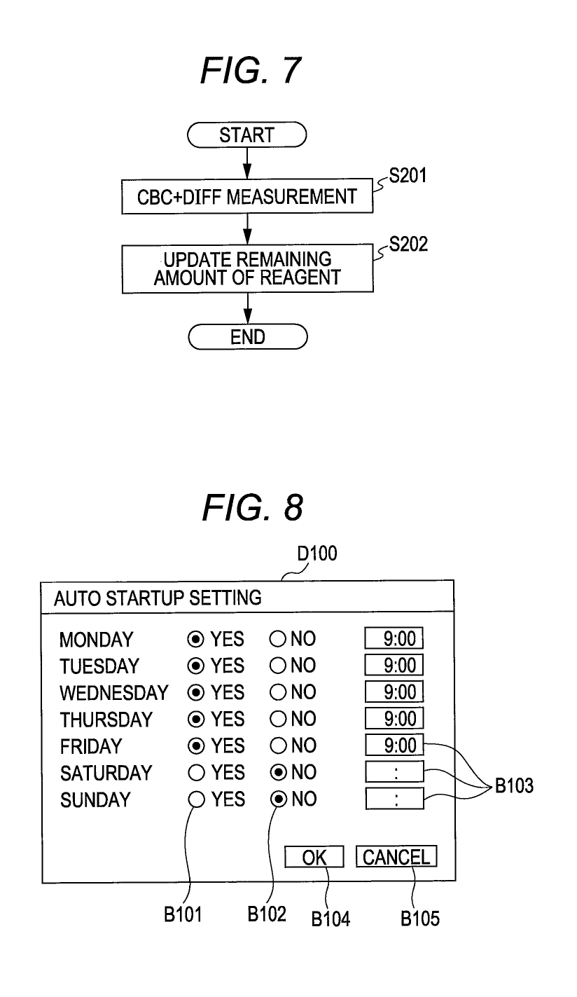

FIG. 7 is a flowchart showing an operation procedure of the sample processing apparatus 1 in the CBC+DIFF measurement. First, the CPU 51a of the information processing unit 5 causes the measurement unit 2 to execute the CBC+DIFF measurement (step S201). In the CBC+DIFF measurement, the hemolytic agent FFD (0.5 mL) is supplied from the hemolytic agent tube FFD-V to the second mixing chamber MC2, and the whole blood sample of the sample tube T is aspirated by a constant amount with the aspirating tube 211 and discharged to the second mixing chamber MC2. The stain fluid FFS is also supplied to the second mixing chamber MC2, and the hemolytic agent FFD is further supplied to the second mixing chamber MC2. When the fluid in the second mixing chamber MC2 is stirred, the red blood cells dissolve into the second mixing chamber MC2, so that the CBC+DIFF measurement specimen in which the white blood cells are stained is created. The CBC+DIFF measurement is then carried out in the WBC detector (optical detector) D3 with the CBC+DIFF measurement specimen as the target. In the CBC+DIFF measurement operation, the charging diaphragm pump DP2 is driven, so that 1.0 mL of the CBC+DIFF measurement specimen is charged, and thereafter, the sheath fluid (diluted solution) EPK is supplied from the EPK accommodating tube EPK-C to the WBC detector. The specimen supplying syringe pump SP2 is driven in such state, and the measurement is carried out in the WBC detector D3.

An output signal (analog signal) output by such WBC detector D3 is converted to a digital signal by an A/D converter (not shown), subjected to a predetermined signal processing by a signal processing circuit (not shown) to convert digital data to measurement data, and the measurement data is transmitted to the information processing unit 5. The CPU 51a of the information processing unit 5 executes a predetermined analyzing process on the measurement data to generate analysis result data including the numerical value data of the NEUT, LYMPH, EO, BASO, MONO, and WBC and stores the analysis result data in the hard disc 51d.

After executing the CBC+DIFF measurement described above, the CPU 51a decrements the remaining amount of the reagent (diluted solution, white blood cell classifying hemolytic agent, and white blood cell classifying stain fluid) used in the CBC+DIFF by one to update the reagent remaining amount information 54b (step S202), and terminates the process.

### Setting of Startup

The startup can be set in the sample processing apparatus 1 according to the present embodiment. The setting of the startup will be hereinafter described.

The setting of the startup is carried out by a startup setting screen. When the operator makes a predetermined input using the input unit 53 of the information processing unit 5, the CPU 51a can cause the display unit 52 to display the startup setting screen. FIG. 8 is a view showing a startup setting screen. In a startup setting screen D100, whether to enable automatic startup can be set, and when enabling the automatic startup, the scheduled time can be set for every day of the week. The automatic startup refers to the operation in which the sample processing apparatus 1 automatically starts when the scheduled time is reached.

The operator selects a radio button B101 corresponding to the day of the week, at which to enable automatic startup, in the startup setting screen D100 with a predetermined operation such as clicking the left button of the mouse to specify the day of the week to carry out the automatic startup. The operator inputs the scheduled time to execute the automatic startup to the input box B103 using the keyboard to set the scheduled time of the automatic startup. If the operator selects the radio button B102, which means not enabling the automatic startup, rather than the radio button B101, the relevant day of the week is set as the day of the week not to carry out the automatic startup. In FIG. 8, the automatic startup is set with respect to each days of the week from Monday to Friday, and the respective scheduled time is set to 9:00. Saturday and Sunday are regular holidays of the facilitate in which the sample processing analysis 1 is installed, and thus are set with “not carry out automatic startup”.

Thereafter, when the operator clicks the button B104 of the startup setting screen D100, the CPU 51a stores the content set when the operator makes an input in the startup setting screen in the hard disc 51d as the setting information 54c, and closes the startup setting screen D100. When the operator clicks the button B105 of the startup setting screen D100, the CPU 51a does not store the content input by the operator in the startup setting screen in the hard disc 51d as the setting information 54c, and closes the startup setting screen D100.

### Stopping Process

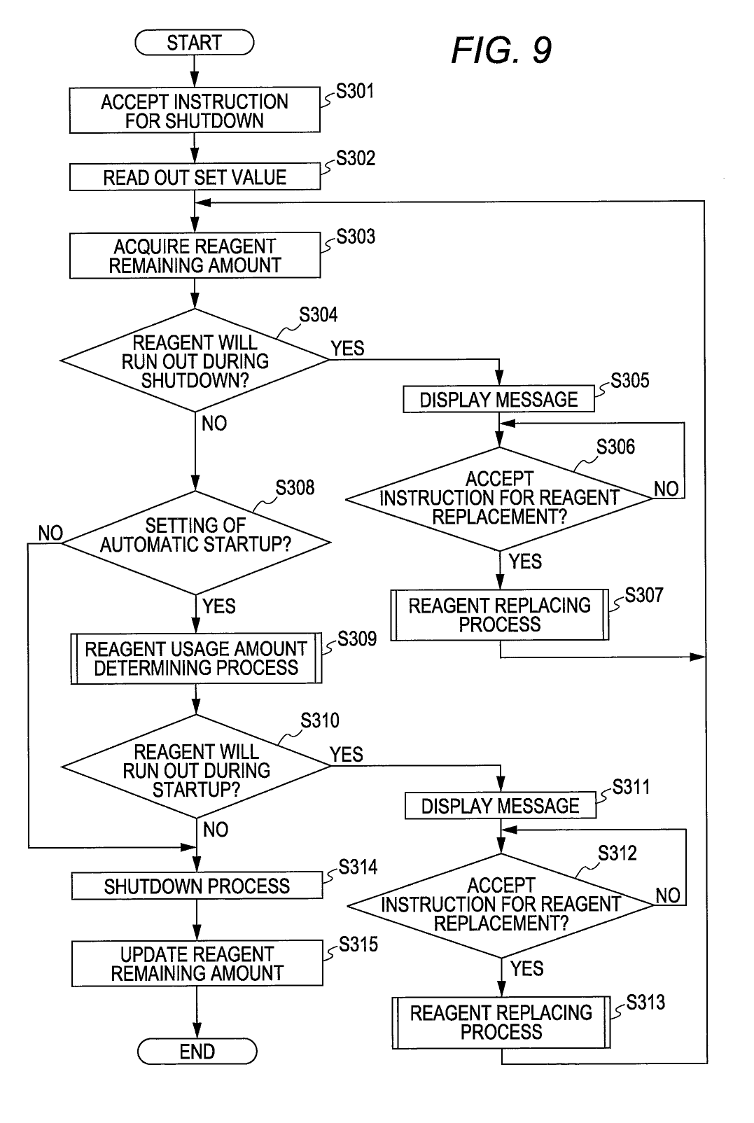

FIG. 9 is a flowchart showing a flow of stopping process of the sample processing apparatus 1 according to the present embodiment. The stopping process of the sample processing apparatus 1 is an operation for having the measurement unit 2 in the stopped state and the information processing unit 5 in the stopped state. In the present embodiment, the stopped state of the measurement unit 2 is a state in which the power supply of the measurement unit 2 is shielded. Further, in the present embodiment, the stopped state of the information processing unit 5 refers to a state in which the computer program 54a started in the information processing unit 5 is terminated (i.e., without storing the operation state immediately before the arrest of the function) and the operating system is also terminated.

The stopping operation of the measurement unit 2 is an operator for having the measurement unit 2 in the stopped state, and the stopping operation of the measurement unit 2 is the shutdown operation of the measurement unit 2 in the present embodiment. The shutdown operation of the measurement unit 2 is the operation for stopping the measurement unit 2 in a state the sample measurement can be normally carried out when the measurement unit 2 is started the next time, and includes a washing operation of the measurement mechanism 2a and an operation of filling the sheath fluid in a flow path in the measurement mechanism 2a.

The startup operation of the measurement unit 2 is the operation for causing the measurement unit 2 in the stopped state to carry out the normal sample measurement, and includes a washing operation and a blank check operation of the measurement mechanism 2a.

When stopping the sample processing apparatus 1, the operator selects a shutdown button (not shown) in a screen displayed on the display unit 52 through a predetermined operation such as clicking the left button of the mouse to give an instruction of shutdown of the measurement unit 2 to the information processing unit 5 (step S301). The CPU 51a reads out the setting information 54c of the automatic startup from the hard disc 51d when an event of accepting the instruction of shutdown occurs (step S302). The CPU 51a reads out the reagent remaining amount information 54b from the hard disc 51d, and acquires the remaining amount of each reagent (step S303).

The CPU 51a then determines whether or not the reagent ran out in the shutdown operation of the measurement unit 2 (step S304). The shutdown operation includes the washing operation of the measurement mechanism 2a. In this washing operation, the diluted solution is used as the washing fluid. The diluted solution for three measurements is consumed in the shutdown operation. That is, the diluted solution for three measurements needs to be remaining in order to execute the shutdown operation. In the process of step S304, whether or not the remaining amount of the diluted solution acquired in step S303 is for three or more measurements is determined. Determination is made that the reagent will not run out in the shutdown operation if the remaining amount of the diluted solution is for three or more measurements, and determination is made that the reagent will run out in the shutdown operation if the remaining amount of the diluted solution is not for three or more measurements.

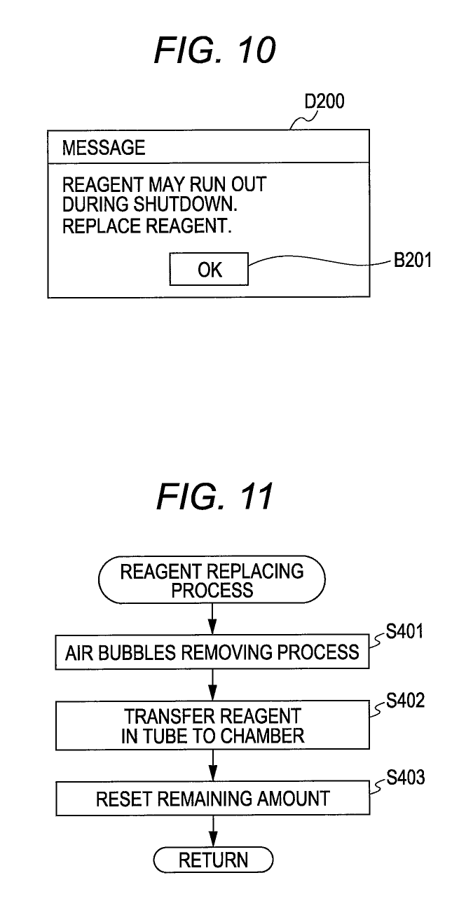

If determined that the reagent will run out in the shutdown operation in step S304 (YES in step S304), the CPU 51a displays a first notification screen, which notifies the operator that there is a possibility the reagent may run out in the shutdown operation, on the display unit 52, and outputs an alarm sound from the speaker 55 (step S305). FIG. 10 is a view showing the first notification screen. A first notification screen D200 includes a message indicating that there is a possibility the reagent may run out in the shutdown operation and that there is a need to replace the reagent. The first notification screen D200 also includes an OK button B201 for instructing the execution of the reagent replacing operation. The button B201 can be selected through a click operation of the left button of the mouse, and the like, where the operator can give an instruction to execute the reagent replacing operation to the sample processing apparatus 1 by selecting the button B201.

In replacing the reagent, the operator changes the reagent tube installed in the sample processing apparatus 1 with a new reagent tube, and causes the barcode printed on the barcode label attached to the new reagent tube to be read by the barcode reader arranged in the sample processing apparatus 1. In the barcode, information such as lot number of the reagent, type of reagent, expiration date, and the like are coded. Thereafter, the operator selects the button B201 of the first notification screen D200 to give an instruction to execute the reagent replacing operation to the sample processing apparatus 1. The CPU 51a determines whether or not the instruction to execute the reagent replacement is accepted (step S306), and again returns to the process of step S306 if the instruction to execute the reagent replacement is not accepted (NO in step S306) and repeats the same to wait for the instruction to execute the reagent replacement. If the instruction to execute the reagent replacement is accepted (YES in step S306), the CPU 51a executes the reagent replacing process (step S307).

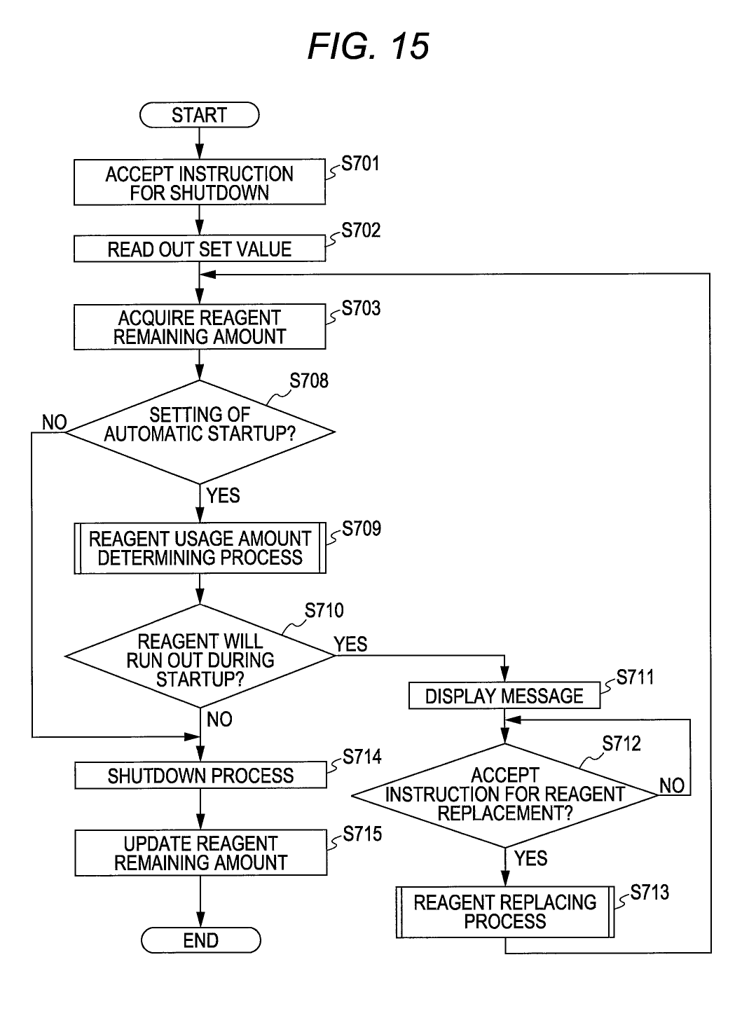

FIG. 11 is a flowchart showing a procedure of a reagent replacing process in S307 and S313 of FIGS. 9, and S713 of FIG. 15. In the reagent replacing process, the CPU 51a first controls the measurement unit 2 and changes the reagent (reagent to be replaced) in the flow path with a new reagent to remove the reagent filled in the flow path of the measurement mechanism 2a (step S401). If the reagent to be replaced is the diluted solution, the CPU 51a aspirates the diluted solution from the reagent tube of the diluted solution that is newly installed, and transfers a predetermined amount of the diluted solution in the reagent tube EPK-V to the diluted solution chamber EPK-C (step S402). The CPU 51a then resets the remaining amount of the reagent (only reagent to be replaced) of the reagent remaining information 54b (step S403), and returns the process to the callout address of the reagent replacing process in the main routine.

After such reagent replacing process is finished, the CPU 51a returns the process to step S303, and again acquires the remaining amount information of the reagent.

If determined that the reagent will not run out in the shutdown operation in step S304 (NO in step S304), the CPU 51a references the setting information 54c read out in step S302 and determines whether or not the automatic startup is set (step S308). If the automatic startup is set (YES in step S308), the CPU 51a executes a reagent usage amount predicting process (step S309).

FIG. 12 is a flowchart showing a procedure of a reagent usage amount determining process in S309 of FIG. 9 and S709 of FIG. 15. In the present embodiment, the usage amount of the reagent changes according to the time from the shutdown operation of the measurement unit 2 to the next startup operation. The reagent usage amount determining process is a process for determining the usage amount of the reagent in the next startup operation.

In the reagent usage amount determining process, the CPU 51a first acquires the current time from the internal clock 51i (step S501), and calculates a time SP from the relevant time to the scheduled time of the next startup operation (step S502).

The CPU 51a determines whether or not the time SP is within 24 hours (step S503). If the time SP is within 24 hours (YES in step S503), the CPU 51a sets “1” to the parameter RT indicating the number of washing (step S504), and proceeds the process to step S508.

If the time SP exceeds 24 hours (NO in step S503), the CPU 51a determines whether or not the time SP is within three days (step S505). If the time SP is within three days (YES in step S505), the CPU 51a sets “3” to the parameter RT (step S506), and proceeds the process to step S508.

If the time SP exceeds three days (NO in step S505), the CPU 51a sets “5” to the parameter RT (step S507), and proceeds the process to step S508.

In step S508, the CPU 51a stores the parameter RT in the hard disc 51d (step S508), and returns the process to the callout address of the reagent usage amount determining process in the main routine.

After the reagent usage amount determining process is completed, the CPU 51a determines whether or not the reagent will run out in the next startup operation (step S310). The startup operation includes the washing operation and the blank check operation of the measurement mechanism 2a. The blank check operation is an operation of causing the measurement unit 2 to execute the measurement operation that does not use a sample, and having the CPU 51a perform the analysis process on the obtained measurement result to obtain the analysis result of each measurement item of RBC, PLT, HGB, NEUT, LYMPH, EO, BASO, MONO, and WBC. In the startup operation, the washing operation corresponding to the number of times determined in the reagent usage amount determining process is carried out, and the diluted solution is used as the washing fluid in the relevant washing operation. In the blank check operation, the reagent of an amount corresponding to the RBC/PLT measurement, the HGB measurement, and the CBC+DIFF measurement, one measurement each, is consumed. In the process of step S310, whether or not the remaining amount of the reagent acquired in step S303 is present by greater than or equal to the amount of reagent consumed in the shutdown operation and the next startup operation is determined. Determination is made that the reagent will not run out in the startup operation if the remaining amount of the reagent is present by greater than or equal to the usage amount of the reagent in the shutdown operation and the startup operation, and determination is made that the reagent will run out in the next startup operation if the remaining amount of the reagent is present by less than the usage amount of the reagent in the shutdown operation and the startup operation.

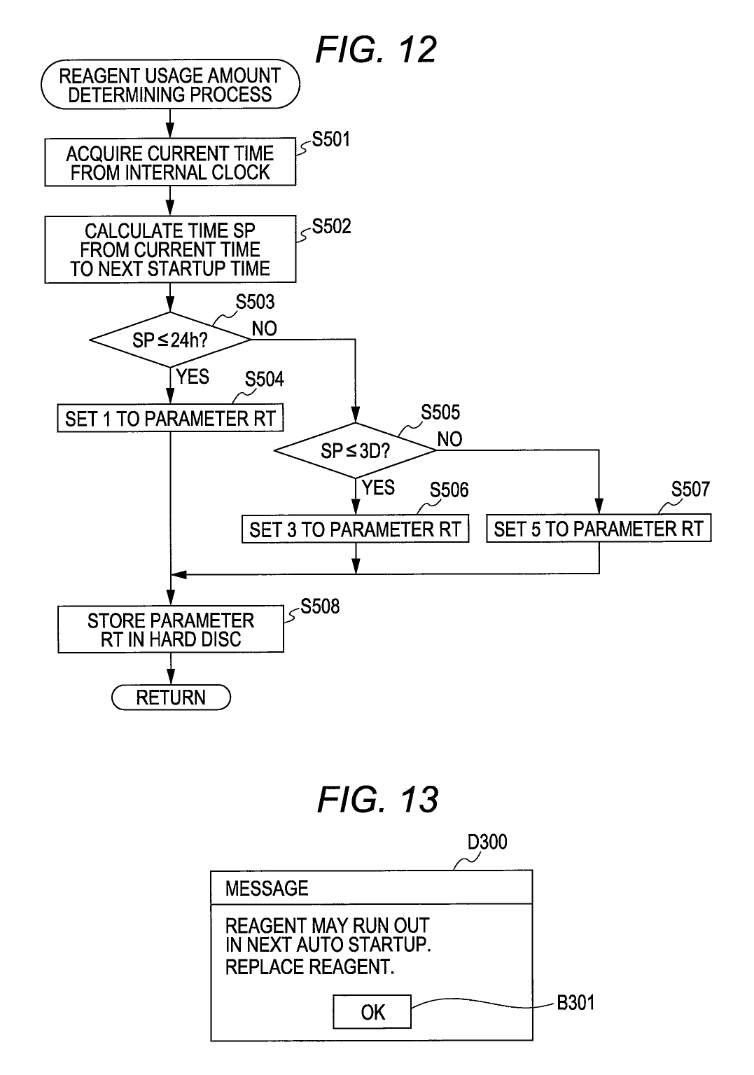

If determined that the reagent will run out in the next startup operation in step S310 (YES in step S310), the CPU 51a displays a second notification screen, which notifies the operator that there is a possibility the reagent may run out in the next startup operation, on the display unit 52, and outputs an alarm sound from the speaker 55 (step S311). FIG. 13 is a view showing the second notification screen. A second notification screen D300 includes a message indicating that there is a possibility the reagent may run out in the next startup operation and that there is a need to replace the reagent. The second notification screen D300 also includes an OK button B301 for instructing the execution of the reagent replacing operation. The button B301 can be selected through a click operation of the left button of the mouse, and the like, where the operator can give an instruction to execute the reagent replacing operation to the sample processing apparatus 1 by selecting the button B301.

The operator changes the reagent tube installed in the sample processing apparatus 1 with a new reagent tube, and causes the barcode printed on the barcode label attached to the new reagent tube to be read by the barcode reader arranged in the sample processing apparatus 1. Thereafter, the operator selects the button B301 of the second notification screen D300 to give an instruction to execute the reagent replacing operation to the sample processing apparatus 1. The CPU 51a determines whether or not the instruction to execute the reagent replacement is accepted (step S312), and again returns to the process of step S312 if the instruction to execute the reagent replacement is not accepted (NO in step S312) and repeats the same to wait for the instruction to execute the reagent replacement. If the instruction to execute the reagent replacement is accepted (YES in step S312), the CPU 51a executes the reagent replacing process (step S313). After such reagent replacing process is finished, the CPU 51a returns the process to step S303, and again acquires the remaining amount information of the reagent.

If determined that the reagent will not run out in the next startup operation in step S310 (NO in step S310) or if the automatic startup is not set in step S308 (NO in step S308), the CPU 51a causes the measurement unit 2 to execute the shutdown operation (step S314). Specifically, the washing of the first mixing chamber MC1, the second mixing chamber MC2, the flow path in the measurement mechanism 2a, and the detectors D1 to D3 is carried out with the diluted solution. After the washing by the sheath fluid is completed, the flow path in the measurement mechanism 2a is filled with the sheath fluid.

After the shutdown operation of the measurement unit 2 is completed, the CPU 51a decrements the remaining amount of the reagent (diluted solution) used in the shutdown operation by three measurements to update the reagent remaining amount information 54b (step S315), and terminates the process. The power supply of the measurement unit 2 and the sample transport unit 4 is then shielded, and the information processing unit 5 is in the stopped state.

### Starting Process

The starting process of the sample processing apparatus 1 will now be described. The starting process of the sample processing apparatus 1 is an operation of having the measurement unit 2 in the stopped state to the start state and resuming the operation of the information processing unit 5 from the stopped state.

The start state of the measurement unit 2 is a state in which the measurement unit 2 can carry out the normal sample measurement. The starting operation of the measurement unit 2 is an operation for having the measurement unit 2 in the start state, and is the startup operation of the measurement unit 2 in the present embodiment. In the present embodiment, the startup operation of the measurement unit 2 is the operation for causing the measurement unit 2 in the stopped state to carry out the normal sample measurement, and includes a washing operation and a blank check operation of the measurement mechanism 2a.

When reaching the scheduled time, the sample processing apparatus according to the present embodiment can enable auto startup in which the startup operation of the measurement unit 2 is automatically executed. The auto startup operation will be hereinafter described in detail.

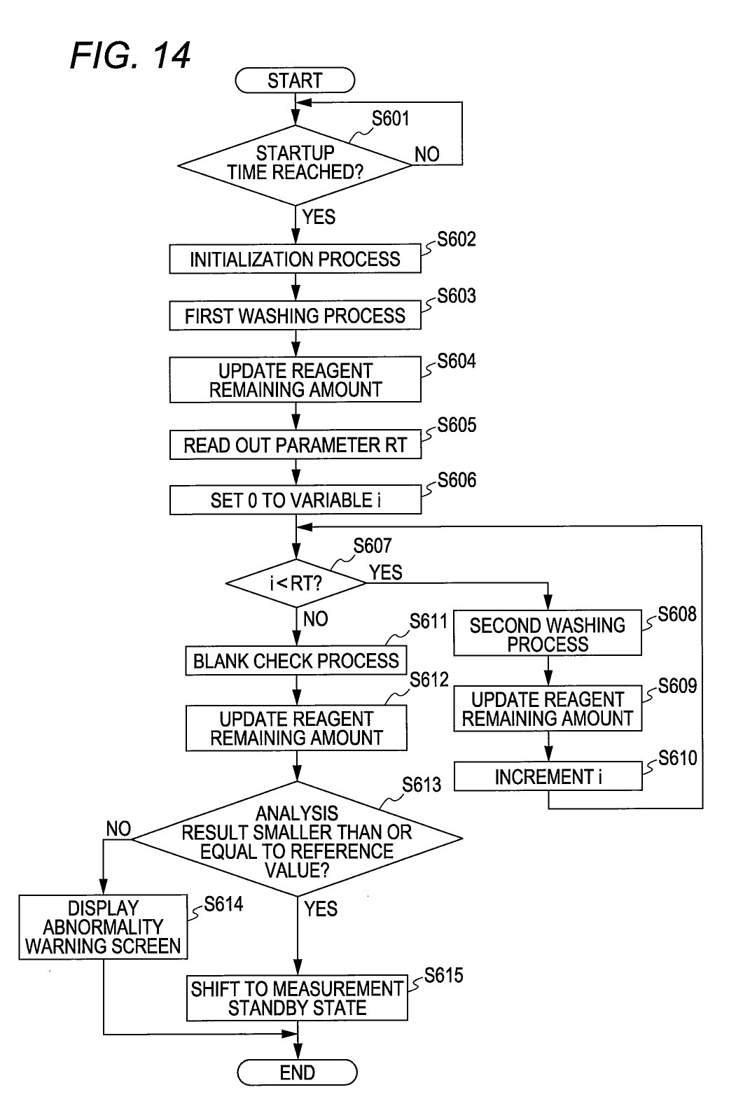

FIG. 14 is a flowchart showing a flow of a starting operation of the sample processing apparatus according to the embodiment. First, the CPU 51a acquires the current time from the internal clock 51i, and determines whether or not the scheduled time of the startup is reached (step S601). If the scheduled time of the startup is not reached (NO in step S601), the CPU 51a again executes the process of step S601, and waits until the scheduled time of the startup is reached.

If the scheduled time of the startup is reached (YES in step S601), the CPU 51a causes the measurement unit 2 to execute the startup operation. The startup operation includes an initial operation, a first washing operation, a second washing operation, and a blank check operation.

When the startup operation is started, the CPU 51a first causes the measurement unit 2 to execute the initial operation (step S602). The initial operation includes supply of power supply, positioning operation of each mechanism portion, warming operation of a heater, and the like. The CPU 51a then causes the measurement unit 2 to execute the first washing operation (step S603). The first washing operation is an operation that is not executed in the measurement operation of the sample in the measurement mechanism 2a (i.e., operation that is not executed in the second washing operation described later), and includes clogging removing operation by applying a pulse voltage to the detectors D1 to D3 and a flashing operation of carrying out the removal of the air bubbles in the detectors D1 to D3. After executing the first washing operation, the CPU 51a decrements the remaining amount of the reagent by the amount used in the first washing operation to update the reagent remaining amount information 54c (step S604).

The CPU 51a then reads out the parameter RT from the hard disc 51d (step S605), and sets “0” to a variable i indicating the repeating number of times of the second washing operation (step S606).

The CPU 51a determines whether or not i is smaller than the washing number of times RT (step S607), and causes the measurement unit 2 to execute the second washing operation (step S608) if i is smaller than the washing number of times RT (YES in step S607).

The second washing operation will be described below. The second washing operation is a measurement operation that does not use a sample. In other words, in the second washing operation, the air is aspirated instead of the sample by the aspirating tube 211 in the aspirating operation of step S102 described above, and then the operations similar to the CBC+DIFF measurement, the RBC/PLT measurement, and the HGB measurement are executed. In one second washing operation, a washing sequence including the CBC+DIFF measurement, the RBC/PLT measurement, and the HGB measurement that do not use the sample (hereinafter referred to as “blank measurement”) is executed once.

In the measurement unit 2 of the sample processing apparatus 1, the diluted solution (sheath fluid) is filled in the flow path shown in FIG. 3A and FIG. 3B on a constant basis since accurate quantitative determination of the sample and the reagent cannot be carried out if air is mixed. This is also the case when the sample processing apparatus 1 is not started. That is, in the shutdown operation, the sheath fluid is filled into the flow path of the measurement mechanism 2a and such state is maintained until the next starting. The sheath fluid is filled in the flow path of the measurement mechanism 2a, but air bubbles generate in the sheath fluid filled in the flow path if the period from the previous shutdown to the power ON is a long period. Furthermore, such air bubbles generate in great amount the longer the period (period from shutdown to startup) in which the sample processing apparatus 1 is stopped. If the air bubbles are remained in the flow path when measuring the sample, the air bubbles may mix into the measurement specimen or the sheath fluid and an accurate measurement may not be carried out. Thus, the air bubbles need to be removed before the start of the measurement of the sample.

In the first mixing chamber MC1 and the second mixing chamber MC2, the fluid such as the sheath fluid is removed after the measurement or after the washing to be in an empty state. Therefore, the first mixing chamber MC1 and the second mixing chamber MC2 are empty in a state the sample processing apparatus 1 after the shutdown is stopped. Immediately after the shutdown, the inner surfaces of the first mixing chamber MC1 and the second mixing chamber MC2 are wet from the attachment of the sheath fluid, and the like, but the inner surfaces of the first mixing chamber MC1 and the second mixing chamber MC2 become dry if the period in which the first mixing chamber MC1 and the second mixing chamber MC2 are not used becomes a long period and dirt, in which components of the sheath fluid and the like are crystallized, may remain on the inner surfaces. Such dirt becomes the cause of degradation in the measurement accuracy. Therefore, the inner surfaces of the first mixing chamber MC1 and the second mixing chamber MC2 need to be sufficiently moistened before starting the measurement of the sample.

The blank measurement is carried out to ensure the removal of dirt an air bubbles, and the wet property of the portion to be used in the measurement. That is, the flow path used in the measurement in the measurement mechanism 2a is washed, the dirt and the air bubbles are removed from the relevant flow path, and such flow path can be sufficiently moistened by carrying out the washing through the same operation as the measurement operation.

The CPU 51a decrements the remaining amount of the reagent by the amount used in one second washing operation after executing the blank measurement to update the reagent remaining amount information 54c (step S609). The CPU 51a then increments the variable i by 1 (step S610), and then returns the process to step S607. The repeating number of times of the washing sequence thus changes according to the length of the period SP from the previous shutdown to the startup. That is, the blank measurement is carried out once if the period SP is within 24 hours, the blank measurement is carried out three times if the period SP is between 24 hours and three days, and the blank measurement is carried out five times if the period SP exceeds three days. The repeating number of times of the blank measurement (washing sequence) is increased the longer the period SP, so that the air bubbles generated in great amount in the flow path can be efficiently removed if the period SP is a long period, and the air bubbles generated only by a small amount can be removed and the time of the washing operation can be suppressed if the period SP is a short period.

If i is greater than or equal to the washing number of times RT in step S607 (NO in step S607), the CPU 51a executes the blank check operation (step S611). The blank check operation is an operation same as the blank measurement described above, that is, an operation of causing the measurement unit 2 to execute the measurement operation that does not use a sample, and having the CPU 51a perform the analysis process on the obtained measurement result to obtain the analysis result of each measurement item of RBC, PLT, HGB, NEUT, LYMPH, EO, BASO, MONO, and WBC. The CPU 51a decrements the remaining amount of the reagent by the amount used by the blank check operation to update the reagent remaining amount information 54c (step S612).

The CPU 51a determines whether or not the analysis result obtained by the blank check operation is smaller than or equal to a predetermined reference value (step S613), causes the display unit 52 to display an abnormality warning screen (not shown) (step S614) if the measurement item in which the analysis result exceeds the reference value exists (NO in step S613), and terminates the process. If the analysis result of the blank check of all the measurement items is smaller than or equal to the reference value (YES in step S613), the CPU 51a shifts the state of the measurement unit 2 to the measurement standby state (step S615), and terminates the process.

### Second Embodiment

In the present embodiment, the volume of the diluted solution chamber EPK-C is that for three measurements. In other words, when performing the shutdown operation, the washing operation can be carried out with only the diluted solution stored in the diluted solution chamber EPK-C. If the diluted solution is used from the diluted solution chamber EPK-C, the diluted solution is supplied from the reagent tube EPK-V to the diluted solution chamber EPK-C, and a state in which the diluted solution chamber EPK-C is full is maintained. Therefore, in the present embodiment, the reagent will not run out in the shutdown operation if the diluted solution chamber EPK-C is full with the diluted solution when an instruction to execute the shutdown operation is given. That is, whether or not the reagent will run out in the shutdown operation does not need to be determined in the stopping process.

The other configurations of the sample processing apparatus according to the present embodiment are similar to the configurations of the sample processing apparatus according to the first embodiment, and thus the same reference numerals are denoted on the same configuring elements and the description thereof will be omitted.

The operation of the sample processing apparatus according to the present embodiment will now be described. FIG. 15 is a flowchart showing a flow of the stopping process of the sample processing apparatus according to the present embodiment. When stopping the sample processing apparatus 1, the operator selects a shutdown button in a screen displayed on the display unit 52 through a predetermined operation such as clicking the left button of the mouse to give an instruction of shutdown to the information processing unit 5 (step S701). The CPU 51a reads out the setting information 54c of the automatic startup from the hard disc 51d when an event of accepting the instruction of shutdown occurs (step S702). The CPU 51a reads out the reagent remaining amount information 54b from the hard disc 51d, and acquires the remaining amount of each reagent (step S703).

The CPU 51a references the setting information 54c read out in step S702, and determines whether or not the automatic startup is set (step S708). If the automatic startup is set (YES in step S708), the CPU 51a executes a reagent usage amount predicting process (step S709), and then executes the processes after step S710. If the automatic startup is not set (NO in step S708), the CPU 51a causes the measurement unit 2 to execute the shutdown operation (step S714). The processes of steps S708 to S715 are similar to the processes of steps S308 to S315 described in the first embodiment, and thus the description thereof will be omitted.

With the configuration described above, the sample processing apparatuses according to the first and second embodiments can prevent the reagent from running out in the startup operation. Furthermore, the operation can be prevented from being interrupted due to lack of reagent in the middle of the startup operation by having the operator replace the reagent according to the second notification screen, so that the startup operation of the sample processing apparatus 1 can be rapidly completed, and the sample process can be rapidly started.

While executing the automatic startup operation, the operator may not be near the sample processing apparatus. If the reagent runs out in the startup operation in such a case, the reagent cannot be replaced until the operator comes near the sample processing apparatus, and hence it is left for a long time in a state the sample process cannot be carried out. According to the sample processing apparatuses of the first and second embodiments, the operator is called to attention that the reagent may run out in the startup operation if reagent is assumed to run out in the startup operation after the operator, who is near the sample processing apparatus, gives an instruction to execute the shutdown operation. Thus, if the reagent is assumed to run out in the startup operation, the operator can reliably replace the reagent beforehand, so that the reagent is prevented from running out in the middle of the automatic startup operation.

If set not to carry out the automatic startup operation, the operator, who turned ON the power, is assumed to be near the sample processing apparatus during the startup operation. In this case, the operator can immediately replace the reagent even if the reagent runs out in the startup operation. According to the sample processing apparatuses of the first and second embodiments, determination is made on whether or not the reagent will run out in the next startup operation only if set to carry out the automatic startup operation, and determination is not made on whether or not the reagent will run out in the next startup operation if set not to carry out the automatic startup operation. Therefore, determination is made on whether or not the reagent will run out only if there is a high possibility the reagent will not be immediately replaced when the reagent runs out in the next startup operation, and thus the operation of the sample processing apparatus can be efficiently carried out.

According to the sample processing apparatuses of the first and second embodiments, the time of the washing operation in the startup operation is made long the longer the time from the shutdown to the next startup. When the time from the shutdown to the next startup is long, sufficient washing is carried out, so that the degradation in the measurement accuracy caused by the attachment of air bubbles, dirt, and the like can be suppressed. When the time from the shutdown to the next startup is short, simple washing is carried out, so that the waiting time until a state the sample can be measured can be shortened. Since the number of executions of blank measurement, which is the washing sequence, is adjusted according to the time from the shutdown to the next startup, the control program for a plurality of washing operations does not need to be separately provided, and the increase in load in designing the program can be suppressed. Furthermore, since the washing sequence is a blank measurement, the flow path used in the measurement of the sample related directly to the measurement accuracy can be reliably washed.

### Other Embodiments

In the first and second embodiments, the sample processing apparatus 1 is a multi-item blood cell analyzer, but is not limited thereto, and the present invention can be applied on various sample processing apparatuses. For instance, in other sample processing apparatuses such as the blood coagulation measurement apparatus, the immune analyzer, the biochemical analyzer, the urine analyzer, and the blood smear creating apparatus, whether or not consumable goods will run out in the next startup operation is determined when an instruction to execute the shutdown operation is given. The lack of the consumable goods in the next startup operation can be predicted for the consumable goods other than the reagent by the type of sample processing apparatus. For instance, in the blood coagulation measurement apparatus and the biochemical analyzer, the lack of the consumable goods in the startup operation is predicted for a disposable cuvette containing a measurement specimen in which the sample and the reagent are mixed. In the immune analyzer, the lack of the consumable goods in the startup operation is predicted for a disposable pipette tip to be attached to a dispensing nozzle for aspirating the sample in addition to the pipette. Furthermore, in the blood smear creating apparatus, the lack of the consumable goods in the startup operation is predicted for a slide glass for smearing the blood. The cuvette and the pipette tip are not used in the washing operation in the startup operation but are used in the blank check measurement in the startup operation.

In the first and second embodiments described above, a configuration of adjusting the length (number of times) of the washing operation according to the time from the shutdown operation to the next startup operation has been described, but this is not the sole case. The same washing operation may be executed on a constant basis in the startup operation irrespective of the time from the shutdown operation to the next startup operation. In this case, the same amount of reagent is consumed on a constant basis in the startup operation, and thus the same reagent usage amount in the startup operation can be used every time and the lack of reagent in the next startup operation can be predicted. In this case, the lack of reagent in the next startup operation can be predicted even if the automatic startup is not set.

In the first and second embodiments described above, the configuration in which the automatic startup operation can be set, and the prediction on whether the reagent will run out in the next startup operation is made only if the automatic startup operation is set has been described, but is not limited thereto.

The prediction on whether the reagent will run out in the next startup operation can be made even if the automatic startup operation is not set. In this case, the prediction on whether the reagent will run out in the next startup operation is made at the time of shutdown, in which the measurement is terminated, and hence the operator can replace the reagent before the next startup operation, and can prepare in advance for the replacement of the reagent so that the reagent replacement rapidly finishes in the next startup operation. The operator thus can rapidly start the sample process.

If the automatic startup operation is always carried out (i.e., configuration in which manual startup operation cannot be set) and the instruction of the shutdown operation is received, the prediction on whether the reagent will run out in the next startup operation may be always made. Furthermore, in both cases of when set to carry out the automatic startup operation and when set not to carry out the automatic startup operation, the prediction on whether the reagent will run out in the next startup operation may be made when receiving the instruction of the shutdown operation. When set not to carry out the automatic startup operation, the prediction on whether the reagent will run out in the next startup operation can be made assuming the maximum value of the reagent usage amount in the startup operation (parameter RT=5 in the case of the same startup operation as the first and second embodiments) as the reagent usage amount of the next startup operation, or the prediction on whether the reagent will run out in the next startup operation can be made assuming the minimum value of the reagent usage amount in the startup operation (parameter RT=1 in the case of the same startup operation as the first and second embodiments) as the reagent usage amount of the next startup operation.

In the embodiments described above, a configuration in which the sample processing apparatus 1 includes one measurement unit 2 has been described, but is not limited thereto. The sample processing apparatus may be configured by two or more measurement units and one information processing unit. The configuration in which measurement unit and the information processing unit are separately arranged may not be adopted, and the sample processing apparatus may have the function corresponding to the measurement unit and the function corresponding to the information processing unit in one housing.

In the embodiments described above, the configuration in which the calculation unit such as the CPU is not arranged in the measurement unit 2 and the operation control of the measurement unit 2 is carried out by the CPU 51a of the information processing unit 5 has been described, but is not limited thereto. A control unit including a CPU, a memory, and the like may be arranged in the measurement unit, so that the operation control of the measurement mechanism can be carried out by such control unit.

In the embodiments described above, the measurement unit and the information processing unit are in the stopped state in the stopping process of the sample processing apparatus, but this is not the sole case. In the stopping process of the sample processing apparatus, the measurement unit may be in the stopped state but the information processing unit may not be in the stopped state.

In the embodiments described above, the stopped state of the measurement unit is a state in which the power supply of the measurement unit 2 is cut, but this is not the sole case. The stopped state of the measurement unit may be a pause state of the measurement unit in which operation is carried out in the power saving mode and the measurement of the sample is not carried out. In this case, when the information processing unit accepts the instruction to start the pause operation for having the measurement unit in the pause state instead of the instruction of shutdown, the usage amount of the consumable goods to be consumed in the pause operation and the usage amount of the consumable goods to be consumed in the starting operation for starting the measurement unit from the pause state to a state (standby state) in which the sample can be measured are determined, whether or not the consumable goods will run out in the next starting operation is determined based on the remaining amount of the consumable goods and the determined usage amount of the consumable goods, and the information notifying the operator that the consumable goods will run out is output if determined that the consumable goods will run out. In the embodiments described above, the stopped state of the information processing unit 5 is a state in which the computer program 54a started in the information processing unit 5 is terminated and the operating system is also terminated, but this is not the sole case. The stopped state of the information processing unit 5 may be a state in which the computer program 54a is terminated and the operating system is not terminated.

In the embodiments described above, the stopped state of the information processing unit 5 is a state in which the computer program 54a started in the information processing unit 5 is terminated and the operating system is also terminated, but this is not the sole case. In the present embodiment, it may be a state in which the information processing unit 5 is operated in the power saving mode after storing the information indicating the operation state (operation state immediately before the arrest of the function) at a certain time point of the information processing unit 5 in the RAM 51c, and the computer program 54a and the operating system are not terminated (suspended). It may be a state in which the information processing unit 5 is operated in the power saving mode after storing the information indicating the operation state at a certain time point of the information processing unit 5 in the hard disc 51d, and the computer program 54a and the operation system are not terminated (hibernation). The state in which the information processing unit 5 is operated in the power saving mode and the computer program 54a and the operating system are not terminated is referred to as the pause state of the information processing unit 5. The information processing unit 5 can return to the operation state immediately before the arrest of the function and resume the operation if from the pause state.

In the embodiments described above, the configuration in which all the processes of the computer program 54a are executed by the single computer 5a has been described, but is not limited thereto, and a distributed system in which the processes similar to the computer program 54a are executed in a dispersed manner by a plurality of devices (computers) may be adopted.

In the embodiments described above, the configuration in which the remaining amount of the reagent is shown by the number of measurements indicating how many more times the measurement can be carried out has been described, but is not limited thereto. The volume may be used for the remaining amount of the reagent.

In the embodiments described above, an example of making the prediction on whether the reagent will run out based on the remaining amount of reagent has been described, but is not limited thereto. The prediction on whether the reagent will run out may be made based on whether or not the number of usages of the reagent from when the reagent is replaced is greater than or equal to a predetermined number of times.

In the embodiments described above, the configuration in which the shutdown process is not executed until the reagent replacing process is completed if the first notification screen for notifying the operator that there is a possibility the reagent may run out in the shutdown operation of the measurement unit 2 or the second notification screen for notifying the operator that there is a possibility the reagent may run out in the next startup operation is displayed on the display unit 52 has been described, but is not limited thereto. The shutdown process may be executed even if the reagent replacement is not completed. In this case, the washing by the diluted solution is not carried out in the shutdown operation of the measurement unit 2.

In the embodiments described above, the configuration of displaying the second notification screen for notifying the operator that there is a possibility the reagent may run out in the next startup operation of the measurement unit 2 on the display unit 52 before the execution of the shutdown operation of the measurement unit 2 has been described, but is not limited thereto. The second notification screen may be displayed on the display unit 52 during the execution of the shutdown operation of the measurement unit 2.

What is claimed is:

1. A sample processing apparatus comprising:
a sample processing unit for processing a sample;
a control unit for accepting a stop instruction to stop the operation of the sample processing unit; and
an output unit,
wherein the control unit is configured to control the output unit to output consumable good information related to lack of the consumable good that occurs if the consumable good runs out before completion of next starting of the sample processing unit when accepting the stop instruction.

2. The sample processing apparatus according to claim 1, wherein
the control unit is configured to control the output unit not to output the consumable good information if the consumable good does not run out before the completion of the next starting of the sample processing unit when accepting the stop instruction.

3. The sample processing apparatus according to claim 1, wherein
the control unit may set a scheduled time to execute the starting of the sample processing unit, and
the control unit is configured to start the sample processing unit when the set scheduled time is reached.

4. The sample processing apparatus according to claim 3, wherein
the control unit can set whether or not to automatically start the sample processing unit when the scheduled time is reached.

5. The sample processing apparatus according to claim 4, wherein
the control unit is configured to control the output unit to output the consumable good information when set to automatically start the sample processing unit when accepting the stop instruction; and
the control unit is configured to control the output unit not to output the consumable good information when set not to automatically start the sample processing unit when accepting the stop instruction.

6. The sample processing apparatus according to claim 1, wherein
the consumable good is used in the starting of the sample processing unit; and
the consumable good information includes information related to the lack of the consumable good that occurs before the completion of the next starting of the sample processing unit.

7. The sample processing apparatus according to claim 1, wherein
the control unit is configured to control the output unit to output the consumable good information, which relates to the consumable good that occurs before the completion of the next starting of the sample processing unit, by the output unit based on a usage amount of the consumable good of when having the sample processing unit in a measureable state from the stopped state.

8. The sample processing apparatus according to claim 1, wherein
the control unit is configured to determine a usage amount of the consumable good in the next starting of the sample processing unit when accepting the stop instruction, and control the output unit to output the consumable good information, which relates to the consumable good that occurs before the completion of the next starting of the sample processing unit, based on a remaining amount of the consumable good and the determined usage amount of the consumable good.

9. The sample processing apparatus according to claim 8, wherein the control unit is configured to determine a usage amount of the consumable good necessary until the completion of the next starting operation according to the time from the stopping of the sample processing unit to the scheduled time of the next starting.

10. The sample processing apparatus according to claim 9, wherein
the control unit is configured to determine the usage amount of the consumable good necessary until the completion of the next starting operation greater as the longer the time from the stopping operation of the sample processing unit to the scheduled time of the next starting is.

11. The sample processing apparatus according to claim 1, wherein
the consumable good is used in the stopping of the sample processing unit, and
the consumable good information includes information related to the lack of the consumable good that occurs by the stopping of the sample processing unit.

12. The sample processing apparatus according to claim 1, wherein the control unit is configured to control the output unit to output the consumable good information, which relates to the lack of consumable good that occurs before the completion of the next starting of the sample processing unit, based on a remaining amount of the consumable good and the usage amount of the consumable good in the stopping and the starting of the sample processing unit when accepting the stop instruction.

13. The sample processing apparatus according to claim 12, wherein the control unit is configured to control the output unit to output the consumable good information, which relates to the lack of consumable good that occurs before the completion of the stopping of the sample processing unit, based on a remaining amount of the consumable good and the usage amount of the consumable good in the stopping operation of the sample processing unit when accepting the stop instruction.

14. The sample processing apparatus according to claim 1, wherein the control unit is configured to cause the sample processing unit to execute restocking or replacement of the consumable good after outputting the consumable good information by the output unit.

15. The sample processing apparatus according to claim 1, wherein the control unit is configured to stop the sample processing unit when restocking or replacement of the consumable good is carried out in the sample processing unit after outputting the consumable good information by the output unit.

16. The sample processing apparatus according to claim 1, wherein the consumable good is a washing fluid for washing the sample processing unit, and the starting operation includes a washing operation that uses the washing fluid.

17. The sample processing apparatus according to claim 16, wherein the washing fluid is a diluted solution for diluting the sample.

18. The sample processing apparatus according to claim 1, wherein the sample processing unit carries out one of blood cell counting, blood coagulation measurement, immunoassay, biochemical analysis, urine analysis, or blood smear creation.
